(19) United States
(12) Patent Application Publication
(10) Pub. No.: US 2003/0100492 A1
(43) Pub. Date: May 29, 2003

(54) Proteoglycans and pharmaceutical compositions comprising them

(76) Inventor: Avner Yayon, Siitria (IL)

Correspondence Address: Abelman Frayne & Schwab, 150 East 42nd Street, New York, NY, 10017-5612, US

(21) Appl. No.: 10/149,326

(22) PCT Filed: Dec. 5, 2000
(86) PCT No.: PCT/IL00/00821

(30) Foreign Application Priority Data
Dec. 5, 1999 (IL) 133318

(51) Int. Cl. A61K 48/00; C07K 14/475; A61K 38/18; C12P 21/02; C12N 5/06; C07H 21/04

(52) U.S. Cl. 514/12; 514/44; 530/399; 530/397; 435/69.4; 435/320.1; 435/325; 536/23.5

(57) ABSTRACT

A molecule is provided capable of promoting high affinity binding of a fibroblast growth factor (FGF) to a FGF receptor (FGFR), said molecule being selected from: (i) a recombinant chimeric fusion molecule comprising the extracellular domain of a syndecan or a fragment thereof fused to a tag suitable for proteoglycan purification, said fusion molecule being post-translationally glycosylated to carry at least one chain of a heparan sulfate having at least one highly sulfated domain; (ii) a DNA sequence encoding a chimeric fusion molecule comprising the extracellular domain of a syndecan or a fragment thereof fused to a tag suitable for proteoglycan purification; and (iii) a sugar molecule from a syndecan carrying at least one chain of a heparan sulfate having at least one highly sulfated domain. The compounds may be used for induction of angiogenesis, bone fracture healing, enhancement of wound healing, promotion of tissue regeneration and treatment of ischemic heart diseases and peripheral vascular diseases.

### FIELD OF THE INVENTION

The present invention relates to heparan sulfate proteoglycans, particularly to syndecans, and to their several uses in promotion of tissue-specific cell proliferation, migration and differentiation.

ABBREVIATIONS: FGF, fibroblast growth factor, FGF2, basic FGF; FGF1, acidic FGF; FGFR, FGF receptor; HS, heparan sulfate; HSPG, heparan sulfate proteoglycan; EDHS, endothelial cells derived HSPG; AP, alkaline phosphatase; CHO, chinese hamster ovary; DMEM, Dulbecco's modified Eagle medium; FCS, fetal calf serum; GST, glutathione S-transferase; MBS, m-maleimidobenzoyl-N-hydroxysuccinimide ester; SDS, sodium-dodecyl-sulfate; PAGE, polyacrylamide gel electrophoresis; PMSF, phenylmethylsulfonyl fluoride; KLH, keyhole limpet hemocyanin.

### BACKGROUND OF THE INVENTION

Fibroblast growth factors (FGFs) constitute a family of at least eighteen polypeptides which are mitogenic for cells of mesenchymal and neuroectodermal origin (1). FGFs share 30-60% amino-acid sequence homology and a high affinity for heparin and heparan-sulfates (HS). A crucial role for cell surface HS in growth factors activity was revealed by the finding that high affinity receptor binding of basic FGF (FGF2) is abrogated in chinese hamster ovary cell lines defective in their metabolism of glycosaminoglycans (2) and in sulfate depleted myoblasts (3). Receptor binding and biological activity of FGF2 could be fully restored upon the addition of exogenous heparin. Further studies have established the involvement of heparin and HS in the binding and signal transduction of FGF1, FGF2 and FGF4 both in vitro and in vivo (4-9). Direct interaction of heparin with a specific sequence in the extracellular domain of FGF receptor (FGFR) was also demonstrated and shown to be required for FGFR interaction (10). These findings strongly support the idea that a ternary functional complex containing FGF, FGFR and a heparin like molecule is required for the activation of signal transduction pathways linked to the FGF-FGFR complex.

The basic heparan sulfate proteoglycan (HSPG) structure consists of a protein core to which several linear heparan sulfate chains are covalently attached (11). A few HSPGs were purified to homogeneity, including the large extra-cellular matrix HSPG perlecan (12), the membrane associated glypicans (13) and the integral membrane HSPGs, syndecan, fibroglycan (14), N-syndecan (15) and amphyglycan/ryudocan (13, 16). The last four comprise a family of membrane integral HSPGs and were re-named Syndecan 1-4 (in the above same order) (17). The syndecans share a similar structure that includes a short highly conserved intracellular carboxy-terminal region, a single membrane-spanning domain and an extracellular domain with three to five possible attachment sites for glycosaminoglycans (17). The intracellular conserved region of syndecan-4 was recently shown to interact with Protein kinase C and with phosphatidylinositol 4,5-biphosphate, both of which can direct and regulate the recruitment of syndecan-4 to the cells focal contacts (18-20).

A preliminary survey of several defined and affinity purified species of cell surface HSPGs, isolated from fetal lung fibroblasts, including syndecan-1, syndecan-2, and glypican failed to promote high affinity receptor binding of FGF2 (21). A similar lack of activity was observed with various species of HS isolated from bovine arterial tissue that were characterized for their effect on vascular smooth muscle cell proliferation. Most of these species of HS and HSPGs in fact inhibited, in a dose-dependent manner, the activation of FGF2-receptor binding induced by heparin (21, 22). In contrast, perlecan, the large basement membrane HSPG (12) isolated from human fetal lung fibroblasts, was found to induce high affinity binding of FGF2 to FGFR1 as well as to promote FGF dependent angiogenesis in vivo (23). More recently syndecan-2 isolated from macrophages was found to enhance receptor and biological activity of FGF2 (24).

### SUMMARY OF THE INVENTION

Thus, according to the present invention, binding of fibroblast growth factors (FGFs) to their high affinity receptors is potentiated by heparin or heparan sulfate (HS). As described herein, syndecans, integral membrane heparan sulfate proteoglycans (HSPG), either purified from endothelial cells or when ectopically overexpressed, promote high affinity binding of a FGF to a FGF receptor, particularly of FGF2 and FGF1 to FGF receptor 1. When expressed in mutant cells, deficient in total HS or which specifically lack 2-O-sulfated iduronic acids, syndecans do not support receptor binding of FGF1 or 2. Syndecan-4 was also found to form SDS-resistant dimers, similar to those observed for syndecans-1 and 3, the formation of which we find to be partially dependent on its HS chains.

Genetically engineered, chimeric soluble syndecan-1, -2, -3 and -4 ectodomains fused to human gamma globulin Fc, expressed in 293T cells, were found according to the invention to be post-translationally modified to carry predominantly HS chains which support receptor binding and biological activity of FGF1 and FGF2. Taken together, these results indicate that syndecans can serve as an integral membrane modulator of FGF signaling.

The present invention thus relates to a molecule capable of promoting high affinity binding of a fibroblast growth factor (FGF) to a FGF receptor (FGFR), said molecule being selected from:

(i) a recombinant chimeric fusion molecule comprising the extracellular domain of a syndecan or a fragment thereof fused to a tag suitable for proteoglycan purification, said fusion molecule being post-translationally glycosylated to carry at least one chain of a beparan sulfate having at least one highly sulfated domain;

(ii) a DNA sequence encoding a chimeric fusion molecule comprising the extracellular domain of a syndecan or a fragment thereof fused to a tag suitable for proteoglycan purification; and

(iii) a sugar molecule from a syndecan carrying at least one chain of a heparan sulfate having at least one highly sulfated domain.

The molecule according to the invention may promote high affinity binding of FGF1 and FGF2 to FGFR1, or of FGF9 to FGFR2 and to FGFR3, or of any other FGF to its respective receptor(s).

The extracellular domain according to (i) and (ii) above may be an extracellular domain of any of the syndecans -1, -2, -3 or -4, or a fragment thereof, wherein said extracellular domain or fragment preferably comprises the glycosylation sites of the syndecan molecule. In the case of syndecan-4, the extracellular domain comprises the amino acids 1-145 of syndecan-4, and a fragment thereof comprises at least 75 amino acids of the extracellular domain of syndecan-4.

According to the invention, the syndecan extracellular domain may be fused to any tag suitable for proteoglycan purification including, but not being limited to, glutathione S-transferase (GST) or polyHis, and preferably the Fc region of the human gamma globulin heavy chain.

The post-translational glycosylation occurs when a DNA molecule according to (ii) above is expressed in suitable mammalian cells including, but not being limited to, endothelial, fibroblast, and epithelial cells, such as embryonic kidney cells, ovary cells, e.g. chinese hamster ovary cells (CHO), or aortic endothelial cells. The type of syndecan and/or the type of cells in which the fused molecule is expressed will determine the tissue specificity of the fused molecule.

The glycosaminoglycan chains of syndecans according to (iii) above may be prepared by protease treatment of the syndecan, for example as described in Nader et al., 1987 (27). The heparan sulfate that constitutes the glycosyl chain of the syndecan has, preferably, at least one highly O-sulfated domain of at least 10 sugar units, and is preferably 2-O-sulfated.

Syndecan coding sequences may be obtained by cDNA cloning or by reverse transcriptase PCR cloning by standard methods well known in the art. The desired extracellular domain or fragments thereof can then be excised by restriction enzyme digest or by PCR using appropriate oligonucleotide primers. The so obtained sequences may then be fused to a suitable tag to form the DNA sequences of (ii) above, preferably with the Fc of the immunoglobulin heavy chain, most preferably human IgG1. When expressed as a fusion protein, the ectodomain of the syndecan will usually be cleaved from the fusion partner. This expression may occur in vivo after administration of a DNA sequence of (ii) above, thus making the soluble biologically active extracellular domain of the syndecan available to exert the desired biological activity.

In particular embodiments of the invention, the recombinant chimeric fusion molecule comprises the extracellular domain of syndecan- 1, -2, -3, or -4 fused to the recombinant Fc region of the human gamma globulin heavy chain, carrying at least one chain of a heparan sulfate having at least one highly sulfated domain (Syn1-Fc, Syn2-Fc, Syn3-Fc, Syn4-Fc). In the case of Syn4-Fc, the chimeric molecule may carry 1, 2 or the 3 polysaccharide chains of Syn4.

The chimeric fusion molecule of (i) above, the DNA molecule of (ii) above and the syndecan derived sugar molecule of (iii) above are capable of modulating (both enhancing and inhibiting) heparin-dependent growth factor activity relevant for promoting tissue-specific cell proliferation, migration and differentiation. The growth factor which activity can be modulated by said molecule includes, but is not limited to, a FGF, a vascular endothelial growth factor (VEGF), hepatocyte growth factor (HGF), an epidermal growth factor (EGF) and keratinocyte growth factor (KGF).

The present invention thus further relates to pharmaceutical compositions comprising a molecule (i), (ii) or (iii) of the invention and a pharmaceutically acceptable carrier. This composition can be used for induction of angiogenesis, bone fracture healing, enhancement of wound healing, promotion of tissue regeneration and treatment of ischemic heart diseases and of peripheral vascular diseases, for example for promoting liver regeneration, or for promoting tissue regeneration alter transplantation of myocytes into heart tissues, or after transplantation of cells into brain tissue.

The molecules of the invention can further be used in combination with one or more growth factors such as a FGF, e.g. FGF2, a VEGF, an EGF, HGF and/or KGF. The growth factor may be administered before, together with, or after the molecule of the invention. For example, a molecule of the invention may be administered together with: (a) FGF2 for treatment of heart failure by transplantation of myocytes, or for promotion of tissue regeneration after transplantation of dopaminergic/neuronal cells for example in Parkinson disease; (b) FGF2 and/or VEGF for induction of angiogenesis or for treatment of ischemic heart disease or peripheral vascular disease; (c) HGF for promoting liver regeneration; (d) KGF for enhancement of wound healing.

### BRIEF DESCRIPTION OF THE DRAWINGS

FIG. 1 shows that binding of FGF2 and FGF1 is modulated by purified endothelial derived syndecan-4. Soluble extracellular domain of FR1-AP fusion protein was immunoprecipitated with anti-alkaline phosphatase antibodies and incubated with ¹²⁵I-FGF1 (right panel) or ¹²⁵I-FGF2 (left panel), in the absence or presence of 1 μg/ml heparin, endothelial derived syndecan-4 (EDHS), or the isolated syndecan-4 HS chains. Binding was performed as described under ‘Experimental Procedures’. Bound complexes were extensively washed with low affinity buffer to remove FGFs bound to the HS. The associated radiolabeled FGFs were determined by a gamma-counter. These results represent one out of three independent experiments, carried out in duplicates. Standard error bars are indicated.

FIGS. 2A-2B show overexpression of syndecan-4 in CHO-KI cells. FIG. 2A: Confluent cultures of wild type CHO-KI cells transfected with syndecan-4 cDNA were incubated with specific monoclonal antibodies directed to the extracellular domain of syndecan-4, and detected by radiolabeled anti-mouse antibodies (filled bars). Cells were lysed and counted in a gamma-counter. CHO-KI cells of the identified syndecan-4 positive clones were metabolically labeled with ³⁵S-sulfuric acid for 24 hours and the amount of heparan sulfate associated radioactivity was measured by liquid scintillation (dashed bars) as described under ‘Experimental Procedures’. Each point represents the mean of duplicate determinations. P-parental untransfected cells; E1, E4, E5 & E10-. The isolated positive CHO-KI clones transfected with syndecan-4 cDNA. FIG. 2B: Positive clones of wild type CHO-KI and GAG deficient mutant CHO-745 were extracted as described under ‘Experimental Procedures’ and treated with heparinase-I and -II mixture. Syndecan-4 protein bands were examined by running equal amounts of cell extracts on SDS-PAGE and transferring to a nitrocellulose membrane. Detection was done with P710 anti-syndecan-4 polyclonal antibodies.

FIGS. 3A-3B show binding of FGF2 to FGFR1 on immobilized syndecan-4. Cells of the indicated clones were extracted as described under ‘Experimental Procedures’. Equal amounts of cell extracts were immunoprecipitated with anti-P710 antibodies and incubated with FGF2 (50 ng). FIG. 3A: Proteins were separated on reducing SDS-PAGE containing β-mercaptoethanol, and transferred to a nitrocellulose membrane. FGF2 was detected with the FB-8 monoclonal antibody. Minor amounts of FGF2 were non-specifically bound to the beads (right lane). D-dimers; M-monomers. FIG. 3B: Similar samples were further incubated with FR1-AP and the amount of the bound receptor was estimated by the associated alkaline phosphatase enzymatic activity as described under ‘Experimental Procedures’. Each data point is the mean of duplicate determinations after subtraction of non-specific binding.

FIGS. 4A-4C show expression and metabolic labeling of a soluble secreted Syn4-Fc fusion protein. FIG. 4A: The extracellular domain of syndecan 4 cDNA (black) was subcloned into the CDM7 vector in frame with the Fc portion of human gamma globulin (doted). The BamHI and HindIII sites used for cloning are indicated. FIG. 4B: CDM7-Syn4-Fc plasmid was co-transfected with the pcDNA3 neomycin resistant vector into 293T cells, and positive clones were selected by dot-blot analysis. Conditioned medium collected from these cells was treated with a mixture of Heparinase-I and -III and analyzed by 10% SDS-PAGE. The proteins were transferred to nitrocellulose membrane and detected with horseradish peroxidase conjugated to Protein A. FIG. 4C: Positive 293T clones expressing the Syn4-Fc fusion protein were metabolically labeled with ³⁵S-sulfuric acid and ³H-leucine for 24 hours The conditioned medium was collected and concentrated on Protein-A Sepharose. Equal amounts of radiolabeled syndecan-4 from each of the different clones (Table 1) was separated on a 3-15% gradient SDS-PAGE without or with pre-treatment with heparinase-I and -III (Hepa's). The gel was dried and exposed to X-ray Kodak film for 3 days.

FIGS. 5A-5C show that syn4-Fc promotes the binding and mitogenic response to FGF2 and FGF1. FIG. 5A: High affinity binding of FGF2 and FGF1 to FGFR1. Conditioned media (100 μl) from 293T cells expressing Syn4-Fc or Erb4-Fc, was immobilized on Protein A Sepharose and incubated in the absence or presence of 75 ng of either FGF1 or FGF2. The coupled beads were washed with HNTG, further incubated with FR1-AP for 2 hours and extensively washed. The bound receptor level was determined by the associated AP activity. FIG. 5B: The ability of conditioned media (100 μl) from 293T cells expressing Syn4-Fc either untreated or treated with heparinase-I and -III (Hepa's), to promote binding of FGF2 to FR1-AP, is indicated. FIG. 5C: Syn4-Fc promotes FGF1 dependent mitogenic response of FGFR1 expressing cells. Thymidine incorporation into heparan sulfate deficient (745) CHO cells overexpressing FGFR1. Cells were serum starved for 24 hours and incubated with or without 5 ng/ml FGF1, in the absence or presence of heparin (Hep) or purified Syn4-Fc (Syn4) at the indicated concentrations (μg/ml) for 14 hours. ³H-thymidine (0.5 μCi/ml) was added for 2 hours, and washed. Cells were fixed, washed and dissolved in 0.1 M NaOH. DNA associated radioactivity was measured by liquid scintillation counting. Each data point represents the mean of duplicate determinations. The variations in the duplicates' results did not exceed 10% of the mean value.

FIG. 6 shows syndecan- 1, -2 and -4 Fc specific induction of FGF-FGFR binding. Conditioned media of growth plate derived chicken chondrocytes cells (LSV) expressing the chimeric Syndecans 1, 2, 3 or 4 fused to the human IgG-Fc fragment were incubated with protein A-agarose beads. The beads were then washed with 2M NaCl and incubated with FGF1, FGF2 or FGF9, following by incubation with soluble FGF receptors (FGFR) 1, 2 and 3, fused to human placental alkaline phosphatase. Significant differences in the binding specificity of the different FGF-FGFR complexes exist. Syn-4-Fc promotes the interaction of FGF2 with FGFR1 and FGFR2 but not with FGFR3. Syn-2-Fc promotes the interaction of all tested ligands with FGFR3 but not all other tested interactions. Surprinsingly, Syn-1-Fc a high affinity interaction of FGF2 with FGFR3, which was not observed with the other syndecans or with cells expressing FGFR3.

FIGS. 7A-7B show the effects of 2-O-sulfation on syndecan-4 activity. FIG. 7A: Positive 293T or Pgs-F17 clones expressing the Syn4-Fc fusion protein were metabolically labeled with 35S-sulfuric acid for 24 hours. The conditioned medium was collected and concentrated on Protein A-Sepharose. Equal amounts of radiolabeled syndecan-4 from each of the different clones (Table 1) were separated on a 3-15% gradient SDS-PAGE. The gel was dried and exposed to X-ray Kodak film for 3 days. FIG. 7B: Conditioned media (100μl) from the above clones was adsorbed to Protein-A Sepharose incubated without or with 75 ng of FGF1 or FGF2, as indicated. The coupled beads were washed with HNTG, further incubated with FR1-AP for 2 hours and extensively washed. The bound receptor level was determined by the AP activity. Each data point is the mean of duplicate determinations.

FIG. 8A depicts the nucleotide and amino acid sequences of syndecan-4. The nucleotide sequence of mouse EDHS (syndecan-4 homologue) and its deduced amino acid sequence in one letter code are shown. The single putative transmembrane domain is underlined. The potential glucosaminoglycans attachment sites are indicated by diamonds (⋄). The doted underline indicates the sequence of the peptide P710 used as antigen for antibody preparation. FIG. 8B: Amino acids sequences of mouse syndecan-1 (49), rat syndecan-2 (50), mouse syndecan-3 (14) and mouse syndecan-4 were compared using the GCG pileup program. Black background indicates at least three identical amino acids, and gray background indicates at least three similar amino acids. FIG. 8C: Amino acids sequences of syndecan-14 from mouse (EDHS, FIG. 8A), rat (ryudocan), human (amphiglycan) and chicken were compared using the GCG pileup program. Black background indicates at least three identical amino acids, and gray background indicates at least three similar amino acids.

### DETAILED DESCRIPTION OF THE INVENTION

The involvement of sulfated glycosaminoglycans in high affinity interactions and signaling of FGFs and other heparin binding growth factors is now well documented (2-5, 7-9, 38). A major outstanding question is the identity of the HSPGs that may carry the oligosaccharide domain, which serves to modulate FGF-receptor interactions in vivo.

In the present application, we describe the expression of the mouse homologue of syndecan-4 and its identification as a candidate cell surface modulator of FGF2 and FGF1 receptor binding and activation. Syndecan-4 expressed either as an integral transmembrane proteoglycan or in a soluble secreted form efficiently enhanced high affinity binding of both FGF1 and FGF2. This effect of syndecan-4 was not restricted to FGFR1 but was shown to occur also with FGFR2. These results indicate that syndecan-4 plays an important role in regulating FGF-FGFR binding and signaling in vivo.

Perlecan, the large basement membrane HSPG was previously found to induce high affinity binding and biological activity of FGF2 (23). More recently glypican isolated from rat embryonal myoblasts (39) and syndecan-1 expressed in Raji lymphoma cells (40) were shown to mediate FGF binding and activity. This may imply some functional redundancy with regard to activation of FGFs by multiple, nevertheless discrete, types of HSPGs from the cell surface and the extracellular matrix. Alternatively, there may be a specific effect for each proteoglycan that is at least partially determined by the localization of the proteoglycan in either the extracellular matrix or at the cell surface. Another possibility is that these proteoglycans may act in synergism to enhance specific activation by FGFs. In retinal pigmented epithelial cells, for example, changes in the expression of both plasma membrane proteoglycans and perlecan are correlated with FGF2 mitogenic activity (41). This co-amplification may serve as an example of a coordinated action of cell surface and extracellular matrix activating proteoglycans that act in concert to enhance FGF signaling.

The presence of an activating HSPG on the cell surface may be of special importance for the autocrine activity of FGF. Such an autocrine activity has been proposed to regulate endothelial cell proliferation and to drive autocrine growth in several melanoma cell lines that produce FGF2 and are dependent on endogenous FGF2, in contrast to normal melanocytes (42). Transformation of NIH-3T3 cells by signal peptide containing FGFs has also been suggested to result from an internal autocrine signaling loop (43, 44). A basic characteristic of this autocrine activity is that all components of the signaling complex including the appropriate HSPG should be expressed within the same cell. Syndecan-4 expression is highly abundant in vivo and is found on a variety of cell lines including endothelial, neural, fibroblastic and epithelial cells (45) where it can serve as an integral part of such an FGF autocrine complex.

The effect of syndecan-4 is solely dependent on its HS chains, therefore, eliminating these chains either by heparinase treatment or by expressing the core protein in the Pgs-A745 CHO mutant cell line, completely abolished its effect. The nature and defined structure of the glycosaminoglycan chains could, in principle, be determined by the nature of the core protein carrying these chains or alternatively by the type and differentiation stage of the cells expressing these core proteins. We show here that expression of syndecan-4 (or its ectodomain) in different cell types including endothelial, fibroblast or epithelial cells results in a recombinant proteoglycan that can bind FGF2 and share a similar capability to promote a high affinity interaction with FGFR1. These findings suggest that at least as far as the HS structure is concerned syndecan-4 can promote FGF2 interaction with its receptor in all the cell systems tested so far. A more quantitative analysis, will assess possible cell type differential effects of syndecan-4 on ligand-receptor specificity.

The structural characteristics of heparin required to promote high affinity binding of FGF2 are specific and restricted to highly O-sulfated oligosaccharides of at least 10 sugar units in length (21, 34, 35). Heparin and HS fragments with high affinity for FGF2 and FGF1 were isolated and found to be polymers rich in 2-O-sulpho-α-L-iduronic acid (46, 47). These specific domains of high charge density, while widely distributed in heparin, are rare in HS, where they may be involved in FGF binding and activation. The HS structure determined for syndecan-4 associated HS chains, isolated from endothelial cells, is composed of four highly sulfated, heparin like domains (27). Each of these contains two regions rich in iduronic acid tri- and disulfated disaccharides and tetra- and pentasulfated tetrasaccharids typical of heparin. Moreover, expression of syndecan-4 in cells incapable of proper 2-O-sulfation, results in a proteoglycan that fails to promote FGF2-receptor interaction, supporting the notion that 2-O-sulfated iduronic acid rich domains in HS are crucial for its FGF promoting activity.

Overexpression of syndecan-4 in wild type CHO cells results in self-association of the core protein and the formation of SDS resistant dimers. A similar phenomenon was reported for syndecan-3, where self-association was suggested to be mediated by a unique structural motif in the protein transmembrane domain (33). This domain is highly conserved among, the different syndecans and may, therefore, share a similar function in syndecan-4 as well. No dimers or higher order oligomers of soluble syndecan-4, lacking the transmembrane domain were detected, suggesting that indeed the sequence responsible for self-association reside within the transmembrane or intracellular domain of syndecan-4. Of special interest is the observation that in HS deficient cells these dimers were significantly less prevalent than in wild type CHO cells, where practically all or most of the syndecan-4 is present as core protein dimers. This may imply that the attached polysaccharide chains may actually enhance core protein association and dimerization. The functional consequences of syndecans self-association are not clear. It was suggested that such association might lead to cytoskeletal element coupling. This is supported by experiments demonstrating that antibody-mediated cross-linking of syndecan-1 in well spread Schwann cells, restored co-localization of the proteoglycan with actin filaments and a concomitant redistribution of cellular actin filaments (15).

Another, most likely related finding regarding syndecan-4, is the recent discovery that it is selectively enriched in focal adhesion contacts (48). A role for HSPGs in adhesion was previously suggested, based on the finding that adhesion defective cells have cell surface HSPGs of altered properties (49). The recruitment of syndecan-4 into focal contacts appears to be coordinately regulated by protein kinase-C (18) and phosphatidylinositol 4,5-biphosphate (19, 20). This recruitment involves direct association and phosphorylation of the C-terminus of syndecan-4 (50) and may serve to stabilize this region. FGFR1, like several other receptor tyrosine kinases, is found to be enriched in focal contacts (51). This co-localization of both FGFRs and accessory HSPGs such as syndecan-4 may serve as means for the local amplification of FGF signals. Alternatively, a role for FGF signaling in the stabilization of the focal contact structure can be suggested. In support for such a hypothesis is the observation that syndecan-4 is a primary response gene induced by FGF2 (52). FGF dependent modulation of focal contacts can drastically affect the adhesion and shape properties of the cell, which in turn may contribute to the well known effects of FGF on cell motility, migration and proliferation in a variety of biological processes such as wound healing and angiogenesis.

The invention will now be illustrated by the following non-limiting Examples.

### EXAMPLES

### Experimental Procedures

Material: Heparin was obtained from Hepar Industries (Franklin, Ohio). Recombinant human FGF2 and FGF1 were kindly provided by American Cyanamid Company (Pearl River, N.Y.). Growth factors were iodinated by the chloramine T method as described previously (25). The specific activity was 1.2-1.7×10⁵ cpm/ng and the labeled preparation was stored for up to 3 weeks at −70° C. Heparinase III and I were purchased from Sigma (St. Louis, Mo.). F12 and Dulbecco's modified Eagle's medium (DMEM), calf serum, fetal calf serum (FCS), penicillin, and streptomycin were obtained from Biological Industries (Beit-Haemek, Israel). G418 was purchased from GibcoBRL (Getthersb, Md.). Tissue culture dishes were purchased from Falcon Labware Division, Becton Dickinson (Oxnard, Calif.). Na¹²⁵I and H₂³⁵SO₄ were purchased from Amersham (Buckinghamshire, England). Triton X-100, nonidet P-40, para-nitro-phenyl phosphate, and all other chemicals were of reagent grade, and purchased from Sigma (St. Louis, Mo.). Anti-FGF2 monoclonal antibody, FB-8, was obtained from Sigma (Israel).

Cell lines—Wild type chinese hamster ovary cells (CHO-KI), glycosaminoglycan deficient (Pgs-A745) or 2-O-sulfated heparan deficient mutants (Pgs-F17) were cultured in F12 medium supplemented with 10% FCS. NIH-3T3 cells were cultured in Dulbecco's modified Eagle's medium (DMEM) supplemented with 10% bovine calf serum. Human embryonal kidney cells (293T) were cultured in DMEM supplemented with 10% FCS.

Purification of syndecan-4—Syndecan-4 was isolated from the conditioned medium of rabbit aortic endothelial cells by Sepharose CL-6B gel filtration followed by ion exchange chromatography on DEAE-cellulose as previously described (26, 27). The identity of the purified proteoglycan was confirmed by N-terminal sequencing (26).

Cloning and expression of syndecan-4 cDNA: Two oligonucleotide primers derived from Syndecan-4 sequence were synthesized; the forward primer, EDF. 5′-CCCAAGCTTTGTGCTGTTGGAACCATGG, and reverse primer EDB: 5′-GCGGATCCGCCTCATGCGTAGAACTCG) having Hind III and BamH I restriction sites at their 5′ ends, (underlined), respectively. The primers were used for PCR amplification (35 cycles of 1 min denaturation at 94° C., annealing for 2 min at 48° C., elongation for 1 minute at 72° C.) with several cDNA libraries (from human placenta, human carcinoma, mouse brain, and mouse liver) used as templates. The amplified products were resolved on a 1% agarose gel stained with ethidium bromide. A PCR fragment of the anticipated size (600 bp) amplified from mouse liver cDNA library was digested with Hind III and BamH I and subcloned into pBluescript KS+ (Stratagene, Calif.). The identity of the amplified fragment was determined by sequencing. A λ-zap cDNA library of 14-day mouse embryo (Stratagene, Calif.) was screened using the PCR product as a probe (hybridization and washing at 65° C.). Positive clones were plaque purified and excised into pBluescript KS+ plasmid according to the manufacturer's instructions. Clones were analyzed by PCR with EDF and EDB primers and their homology to the mouse, rat and human syndecan-4 was confirmed by sequence determination. The cloned mouse syndecan-4 is identical to that of the published sequence except for position 135 where alanine is replaced by a valine which is identical to the human amphiglycan sequence in that position (13). The obtained mouse syndecan-4 cDNA was excised from pBluescript KS+ by Xho I and Xba I and subcloned into the same sites of the pLSV mammalian expression vector (28).

Expression of full length syntlecan-4 in CHO cells—Syndecan-4 in the pLSV expression vector was co-transfected into CHO-KI and Pgs-A745 cells, with a selectable neomycin resistance gene, by the calcium phosphate method. Clones were selected in G418 (0.5 mg/ml) and screened for syndecan-4 expression by direct binding of antibodies directed against the extracellular domain of syndecan-4, or by metabolic labeling of cells with ³⁵S-sulfuric acid (150 μCi for 24-48 hours).

Construction and expression Of chimeric soluble Syn-1-Fc, Syn-2-Fc, Syn-3-Fc and Syn-4-Fc—To express soluble syndecan-1, -2, -3, and -4, we used the immunoglobulin chimeric expression vector CDM7 (Invitrogen, Calif.). For example, the extracellular part of syndecan-4 was amplified by PCR (35 cycles of 1 min denaturation at 94° C., annealing for 2 minutes at 56° C. and elongation for 1 minute at 72° C.) using syndecan-4-pBlueScript as template DNA, the EDF forward primer and the reverse primer EDMB: 5′-CGGGATCCTCAGTTCTCTCAAAGATG that contains a BamH I site (underlined). The purified PCR product was cut with Hind III/BamH I and subcloned in frame to a Fc portion (including the hinge region, CH2 and CH3 domains) of human IgG1, in the CDM7 vector, to create the fusion protein Syn4-Fc. The Syn4-Fc plasmid was co-transfected into 293T cells with the neomycin resistance gene, by electroporation using Gene Pulser (Bio-Rad, Calif.) set at 960 μF and 250 V. Individual clones were selected with G418 (0.6 mg/ml) and screened for Fc secretion by dot-blot of conditioned media (100 μl) with horse-radish-peroxidase (HRP) coupled anti-human Fc antibody (Sigma, Israel). The chimeric molecules with syndecan- 1, -2 and -3 were obtained in the same way.

Preparation of anti-syndecan-4 antibodies—Polyclonal antibodies were prepared against a 12 amino acid long peptide (P710), with a sequence identical to the carboxy-terminus of syndecan-4. A cysteine residue was added at the amino-terminus of the peptide and was used for conjugation to keyhole limpet hemocyanin (KLH) (Calbiochem, Calif.) with m-maleimidobenzoyl-N-hydroxysuccinimide ester (MBS) (Pierce, Ill.). The conjugates then served as antigens for immunization of New Zealand white rabbits. After three injections, the animals were bled and the titer and specificity of the antiserum were determined by immunoprecipitation of HSPGs from labeled lysates of human fetal lung fibroblasts and by competition for binding to syndecan-4 by the specific peptide. An IgG fraction was isolated on a Protein A column according to the manufacturer's instructions (Repligen, Mass.).

In vitro binding of FGFRs to FGFs immobilized on syndecan-4. Syndecan-4 was extracted from overexpressing cells in lysis buffer (150 mM NaCl, 20 mM Tris pH 8.0, 1 mM MgCl₂, 0.1 mM ZnCl₂, 0.5% NP-40, 1 μg/ml aprotinin, 1 μg/ml leupeptin, 2 mM PMSF) and cell lysates were clarified by centrifugation. Total cell extracts (100 μg protein) were immunoprecipitated with polyclonal anti-syndecan-4 antibody (P710). Alternatively, Syn4-Fc fusion protein was immobilized directly on Protein A-Sepharose. FGF (50 ng) was bound to immobilized syndecan-4 for 2 hours at 4° C. The beads were washed extensively with HNTG (150 mM NaCl, 10% glycerol, 0.1% Triton-X-100 and 50 mM Hepes pH 7.4) and incubated for 2 hours with conditioned media containing the soluble FGFR1 or FGFR2-alkaline phosphatase (FR1-AP and FR2-AP, respectively) fusion proteins (29-31) followed by a 0.5 M wash to eliminate non specific binding of the receptor to HS. Alkaline phosphatase activity was monitored spectrophotometrically at 405 nm using para-nitro-phenyl phosphate as a substrate, as described (29). The extent of soluble FR-AP binding was determined by measuring alkaline phosphatase activity associated with the beads after extensive washing with HNTG.

Binding of FGFs to immobilized FR1-AP—FR1 -AP and FR2-AP fusion proteins were immunoprecipitated with anti AP antibodies and incubated for 4 hours with ¹²⁵I-FGF1 and ¹²⁵I-FGF2, in the presence or absence of 1 μg/ml heparin, syndecan-4 or HS-chains, in binding buffer (1% BSA and 25 mM Hepes in DMEM). The binding medium was then discarded and the cells were washed twice with binding buffer and once with 0.5M NaCl in 25mM Hepes pH 7.5. High affinity bound FGFs were eluted with a buffer of 1.6 M NaCl in 20 mM Sodium Acetate pH 4.5 and counted in a γ-counter.

³⁵S-sulfate and 3H-leucine labeling of cells—Post-confluent cultures in 24-well plate were incubated for 24-36 hours in the appropriate medium supplemented with 10% fetal bovine serum, containing 20 μCi/ml of H₂³⁵SO₄ or 10 μCi/ml of ³H-leucine. The cells were washed twice with PBS, and scraped in a small volume of lysis buffer. The cell lysates were clarified by centrifugation and the amount of radioactive material in the pellet was measured by liquid scintillation. Alternatively, if soluble Syn4-Fc was labeled, the conditioned medium was collected and the protein was separated on Protein A-Sepharose.

Purification of syndecan-4 from overexpressing cells—IgG fraction of anti P710 antibody was dialysed against 0.1 M NaHCO₃, 0.5 M NaCl pH 8.3 and coupled to activated Sepharose 4B (Pharmacia, Sweden) according to the manufacturer's instructions. The enriched fraction of total HSPGs from KI-E5 cells, obtained by absorption on DEAE-cellulose (Pharmacia, Sweden) was eluted with 1 M NaCl, 0.1% Triton-x-100. The DEAE eluate was diluted 1:3 in double distilled water and loaded on an affinity column. Syndecan-4 was eluted with 0.2 M glycine/HCl pH 2.5 and neutralized immediately with 1 M Tris pH 8.0.

Purification of soluble Syn4-Fc from tie conditioned medium of overexpressing cells—The chimeric proteoglycan was observed on DEAE-cellulose and eluted with 1 M NaCl, 0.1% Triton-x-100. DEAE eluate was diluted 1:5 in double distilled water and loaded on FPL-Q HiTrap mini column (Pharmacia, Sweden). The column was washed with 75 mM Tris/HCl pH 7.3 and proteins were eluted by 0-1 M NaCl gradient in the same buffer. Syn4-Fc eluted at 0.7 M NaCl was detected by dot blot with horseradish-peroxidase coupled anti-human Fc antibody. Purity was determined by SDS-PAGE and silver staining, and both proteins and glycosaminoglycans were quantitated using the Bradford protein assay (Bio-Rad, Calif.) or the dimethylmethylene blue (32), respectively.

### Example 1

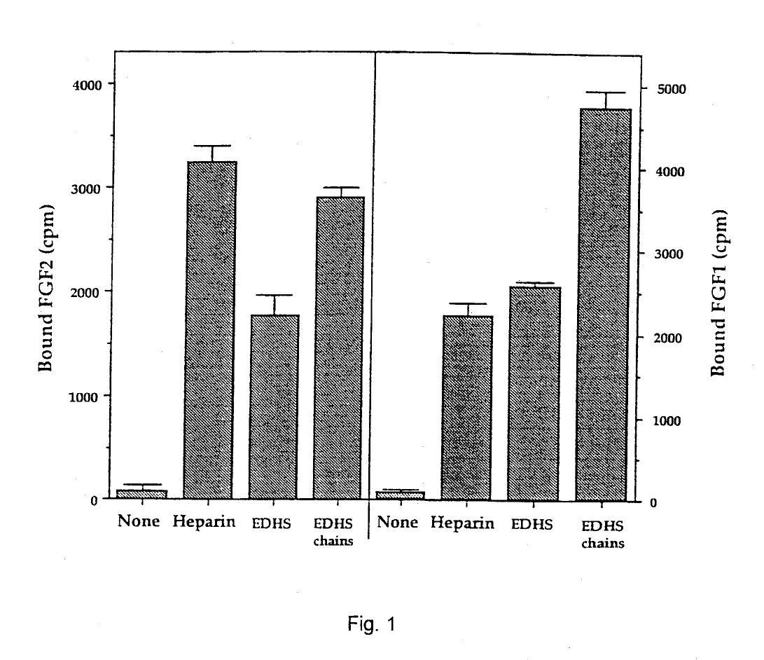

Characterization of an endothelial cell derived syndecan-4 —Syndecan-4 (EDHS in FIG. 1) purified from the conditioned medium of rabbit aortic endothelial cells (26) was examined for its effects on FGF2 binding to FGFR1. In contrast to several other HSPGs (23), a strong induction of FGF2 binding was observed in the presence of syndecan-4 (FIG. 1, left panel). Syndecan-4 also enhanced the binding of FGF1 to soluble FGFR1 (FIG. 1, right panel). Heparan sulfate chains isolated from syndecan-4 by protease treatment (27) had a somewhat stronger effect on the interactions of both ligands with FGFR1 compared to that of the intact proteoglycan (FIG. 1). The effect of purified syndecan-4 is dose dependent with maximal activity at 1 μg/ml while high concentrations (10 μg/ml and higher) inhibit FGF2 binding (not shown), similar to the inhibition observed with high doses of heparin. These results demonstrate that syndecan-4 can efficiently enhance the interactions of FGF1 and FGF2 with their high affinity receptor.

### Example 2

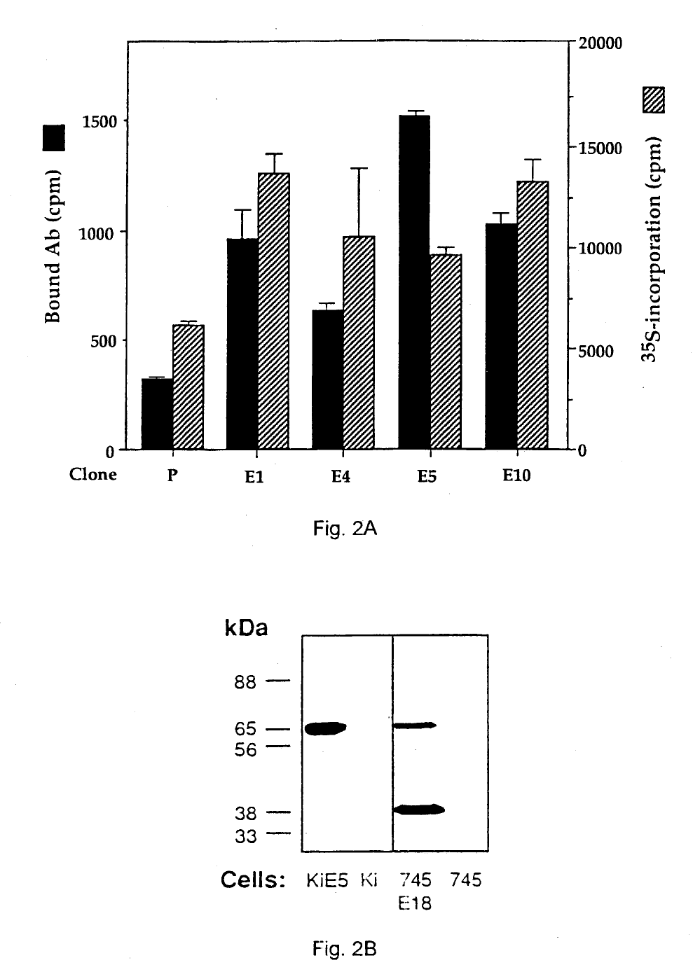

Ectopically expressed mouse syn(lecan-4 is post-translationally modified and expressed as a cell surface HSPG—In order to study the role of syndecan-4 and its HS chains in modulating FGF-receptor interactions, mouse syndecan-4 was overexpressed in CHO-KI and in Pgs-A745-CHO mutant cells deficient in glycosaminoglycans. Positive clones identified by direct binding of monoclonal anti-syndecan-4 antibodies were selected and further tested for expression by immunoblotting (not shown). Measuring radioactive sulfate incorporated into syndecan-4 expressing CHO-KI clones normalized for total syndecan-4 (FIG. 2A) suggests that the ectopically expressed syndecan-4 represent 30-50% of the total HSPG in these cells. Higher levels of expression of syndecan-4 did not lead to increased sulfate labeling (FIG. 2A, clone KI-E5), suggesting that the glycosaminoglycan modifying enzymes may be limiting. Upon heparinase treatment, a single protein band with an apparent molecular mass of 65 kDa was identified in the wild type cells (FIG. 2B, clone KI-E5). In Pgs-A745 clones, on the other hand, two protein bands of molecular mass of 35 and 65 kDa were observed and the 35 kDa form always appeared as the predominant species (FIG. 2B). Similar results were obtained for all positive clones tested (not shown). Clone E10 was chosen for further characterization. The molecular mass of syndecan-4 is 19.25 kDa, as predicted from the cDNA open reading frame. However, both rat and human syndecan-4 were reported to behave anomalously on SDS-PAGE and to migrate at an apparent molecular weight of 33 kDa (16), an abnormality characteristic of all syndecans (17). The high molecular weight form (ca. 65 kDa), even under the denaturing and reducing conditions used, most likely represents denaturation resistant dimers, a phenomenon previously observed for N-syndecanlsyndecan-3 (33).

### Example 3

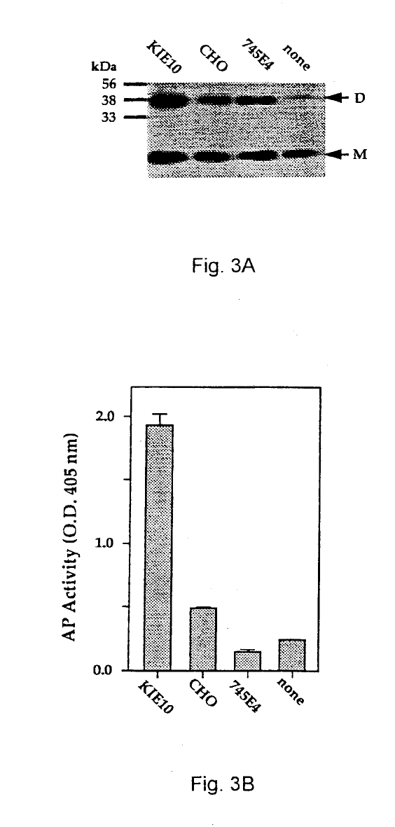

Syndecan-4 binds FGF2 and promotes its binding to FGFR1—Ectopically expressed syndecan-4 efficiently binds FGF2 in vitro as demonstrated by co-precipitation of the proteoglycan and detection by immunoblot with specific anti-FGF2 antibodies (FIG. 3A). Immunoprecipitated syndecan-4 from clone KI-E10 binds approximately 3-fold more FGF2 than syndecan-4 from the CHO-KI parental cells. Immunoprecipitates from either parental pgs-A745 CHO cells or clone 745-E4 bound only minor amounts of FGF2. These results indicate that syndecan-4 associated HS chains are responsible for the binding of FGF2. Moreover, the ratio of dimers to monomers of FGF2 is higher in the KI-E10 IP, indicating that syndecan-4 not only binds FGF2 but can also enhance its dimerization. FGF2 bound to syndecan-4 was also bound with high affinity to FGFR1 (FIG. 3B). Binding of FGF2 to immobilized FGFR-1 was tripled in the presence of syndecan-4 isolated from clone E10 overexpressing the ectopic proteoglycan.

### Example 4

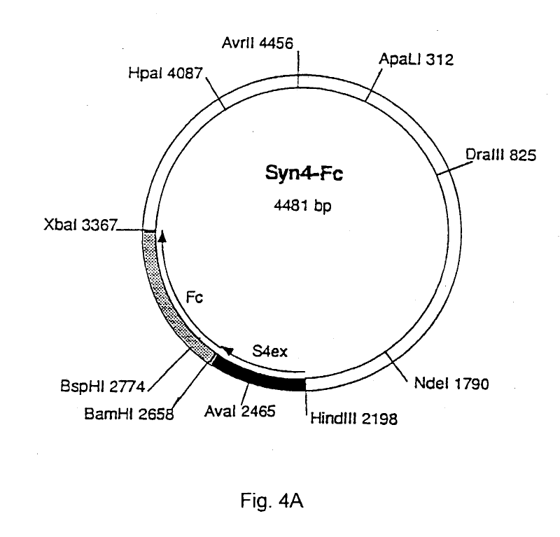

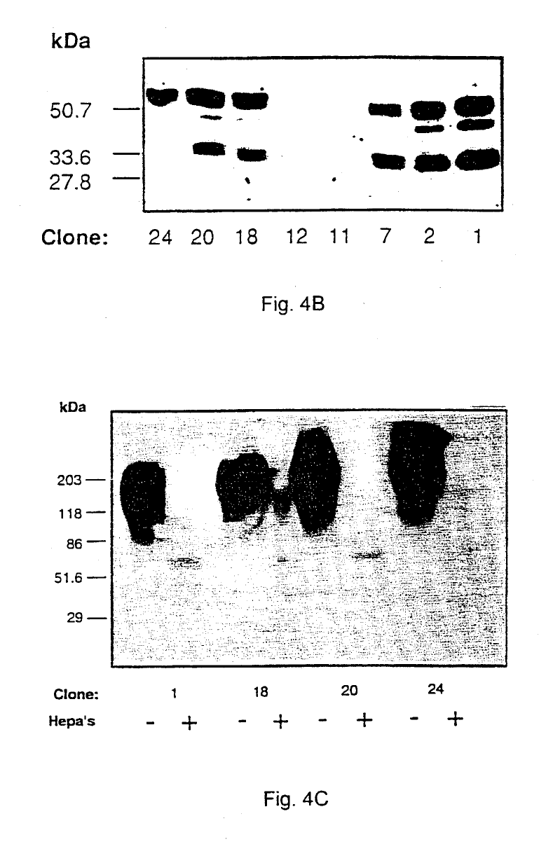

Soluble chimeric syndecan-4 is post-translationaly modified and can modulate FGF-receptor interactions—In order to further study the effects of syndecan-4 on ligand-receptor interactions, a chimeric protein (Syn4-Fc), in which the extracellular part of syndecan-4 was fused to the Fc portion of human IgG1, was generated. The Syn4-Fc was secreted into the conditioned medium of transfected 293T cells, and isolated using Protein A chromatography. SDS-PAGE analysis of conditioned medium from transfected cells, pretreated with heparinase, revealed three protein bands that can be detected by labeled anti-human Fc antibodies (FIG. 4A). A major protein band migrated at ˜60 kDa, somewhat higher than the expected molecular weight of the chimeric fusion protein. This is consistent with the abnormal migration pattern observed for the full length core protein the two additional bands at 33 and 50 kDa represent most likely the Fc portion and a partial degradation product of the fusion protein, respectively.

The Syn4-Fc chimeric protein is post-translationally modified by HS chains as was demonstrated by metabolic labeling with ³⁵S-sulfate (FIG. 4B). Co-labeling with ³H-leucine and H₂³⁵SO₄ enabled us to estimate the relative amount of protein and sugar in the chimeric proteoglycan by measuring radioactivity with the appropriate energy window for each isotope (³⁵S or ³H). The results are summarized in Table 1. The ratio between the two isotopes is 1.54±0.07 for all the samples, indicating that there is a constant ratio of sulfated sugar to protein in all the selected Syn4-Fc secreting clones. Radiolabeled Syn4-Fc from different clones was further analyzed by SDS-PAGE, before and after heparinase treatment (FIG. 4B). The intact proteoglycan appeared as a broad band at 200-220 kDa in all Syn4-Fc preparations tested. Following heparinase treatment the chimeric core protein appeared as a single band with the expected molecular mass of 60 kDa (FIG. 4B). No dimers or higher order oligomers of soluble syndecan-4 were observed suggesting that the transmembrane and/or the short intracellular segments of syndecan-4 may be directly responsible for the spontaneous dimerization observed for the intact proteoglycan.

TABLE 1
Metabolic labeling of Syn-4-Fc fusion protein. Positive clones expressing
Syn4-Fc fusion protein were metabolically labeled with both ³⁵S-sulfuric
acid and 3H-leucine for 24 hours. The condition medium was collected
and immobilized on Protein A-Sepharose. Five percent of each sample was
subjected to liquid scintillation counting (using the ³H and
³⁵S energy windows), and the ³H to ³⁵S ratio was determined.

| Clone | ³⁵S(cpm) | ³H(cpm) | ³⁵SO₄/³H-Leu |
|---|---|---|---|
| 1 | 41113 | 26360 | 1.56 |
| 18 | 14784 | 9725 | 1.52 |
| 20 | 23059 | 15819 | 1.46 |
| 24 | 23114 | 14266 | 1.62 |

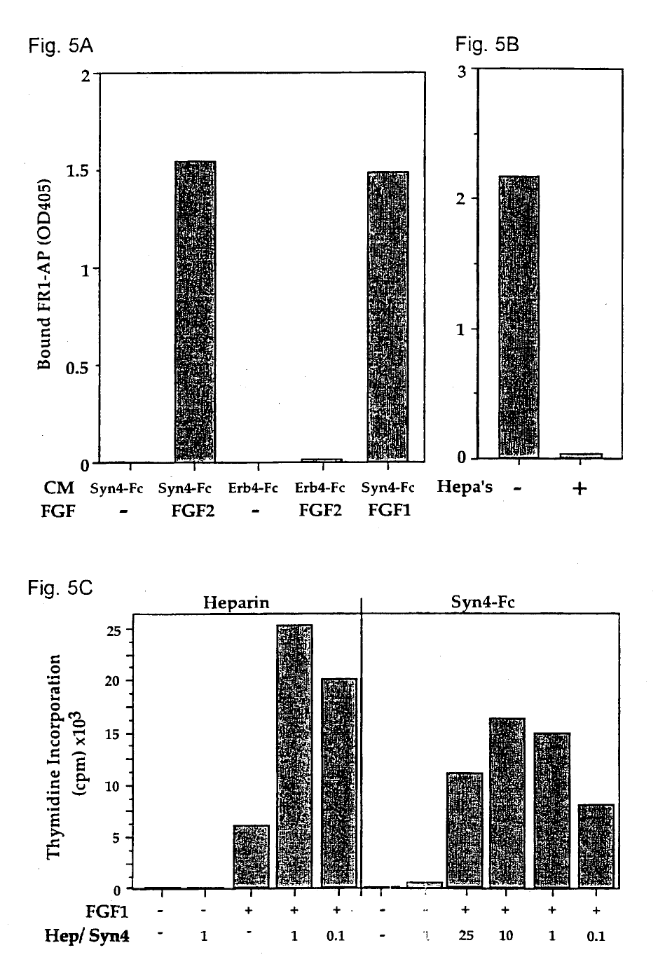

To test the ability of soluble syndecan-4 to promote binding of FGF2 to FGFR1, the conditioned medium from Syn4-Fc expressing cells was absorbed to Protein A beads, incubated with FGF2 and then reacted with soluble FR1-AP. Efficient binding of FR1-AP to immobilized Syn4-Fc-FGF2 complex was observed (FIG. 5A). Binding of FGFR1 also occurred when FGF1 was complexed with Syn4-Fc and was observed only in the presence of the ligands. The activity of Syn4-Fc appeared to be specific to the syndecan-4 part of the fusion protein, as Fc coupled to the extra-cellular part of the Erb4 receptor, used as a control, did not support FR1-AP binding. Treatment of Syn4-Fc with heparinase completely abolished FGF2 binding to FR1-AP (FIG. 5B), indicating that the interaction is via the HS chains and not the core protein. In agreement, no association of FGF2 and soluble FGFR1 with Syn4-Fc produced in HS deficient cells could be detected (not shown). Syn4-Fc was also capable of promoting the direct binding of ¹²⁵I-FGF2 to soluble FGFR2-AP (not shown).

### Example 5

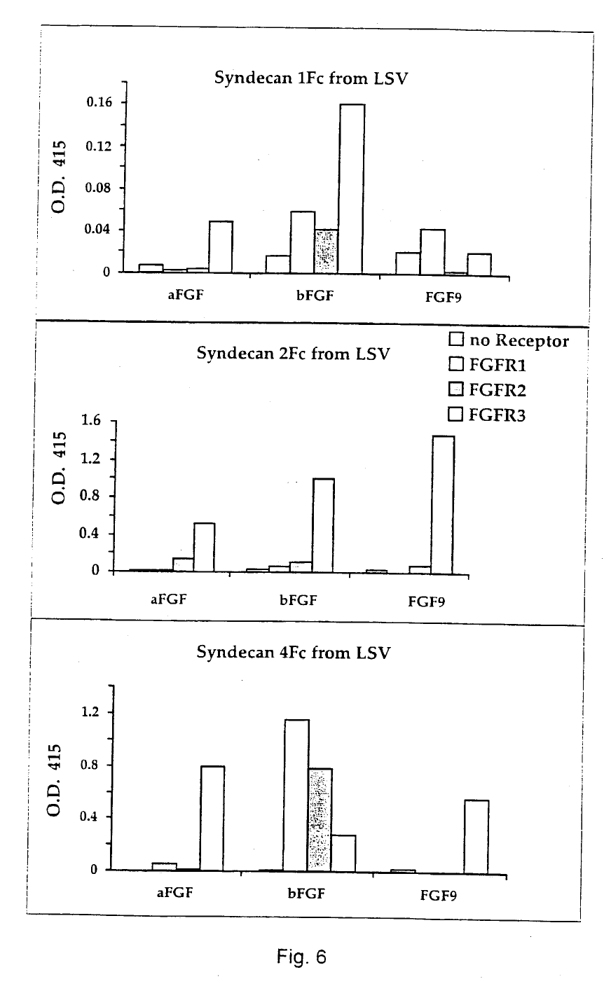

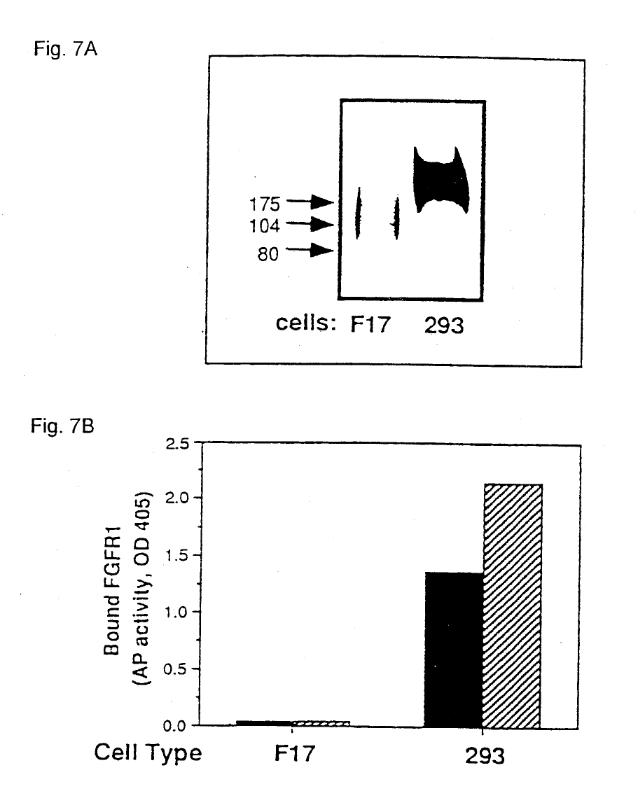

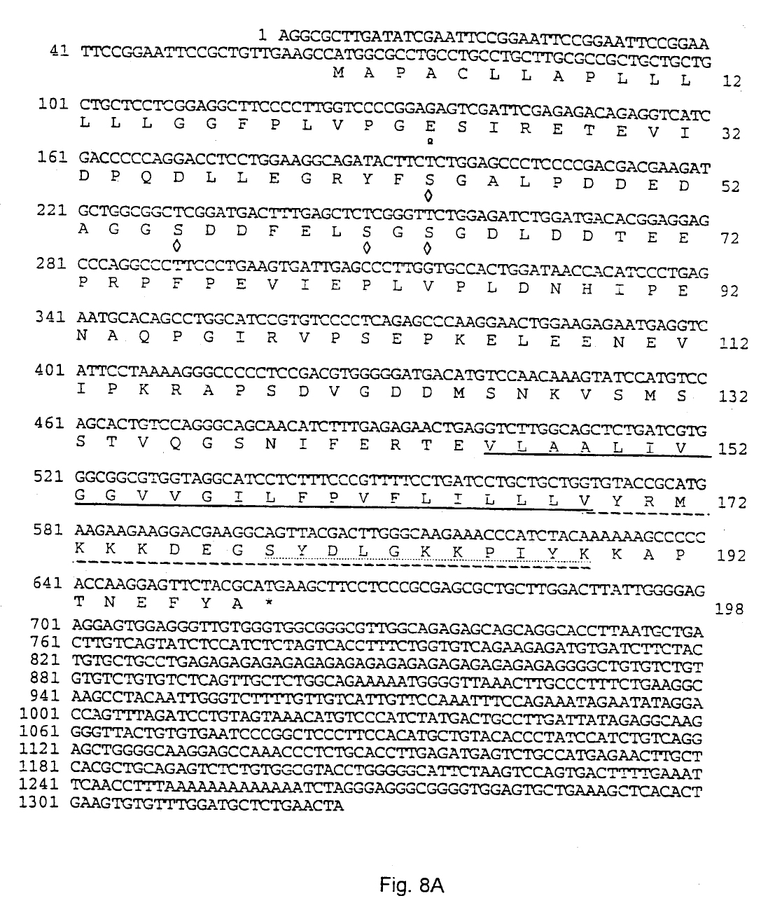

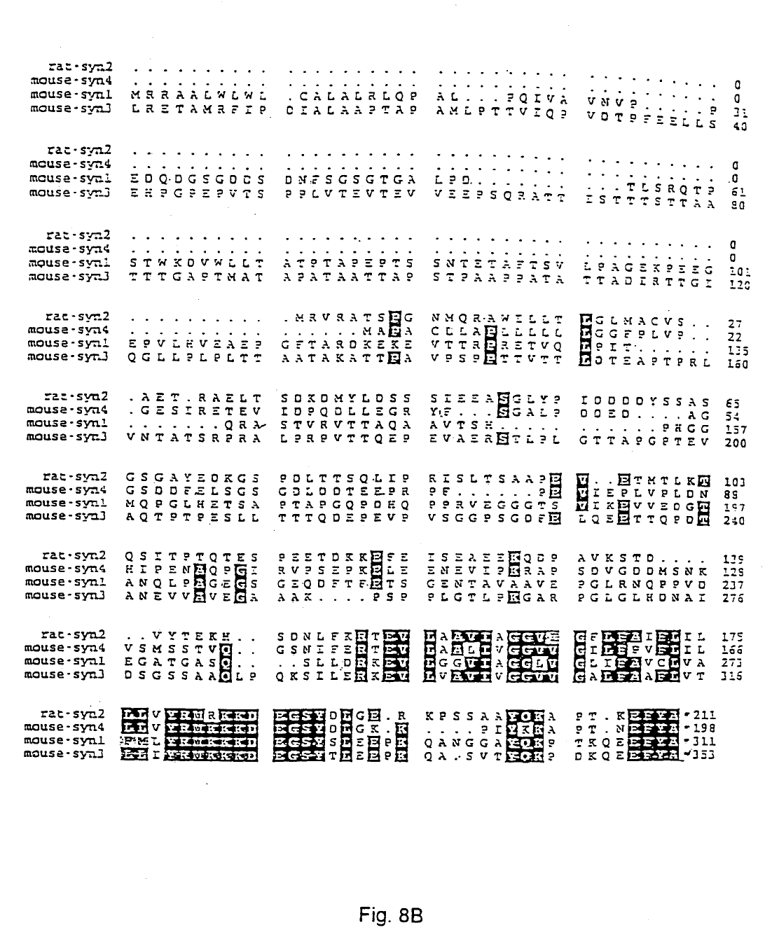

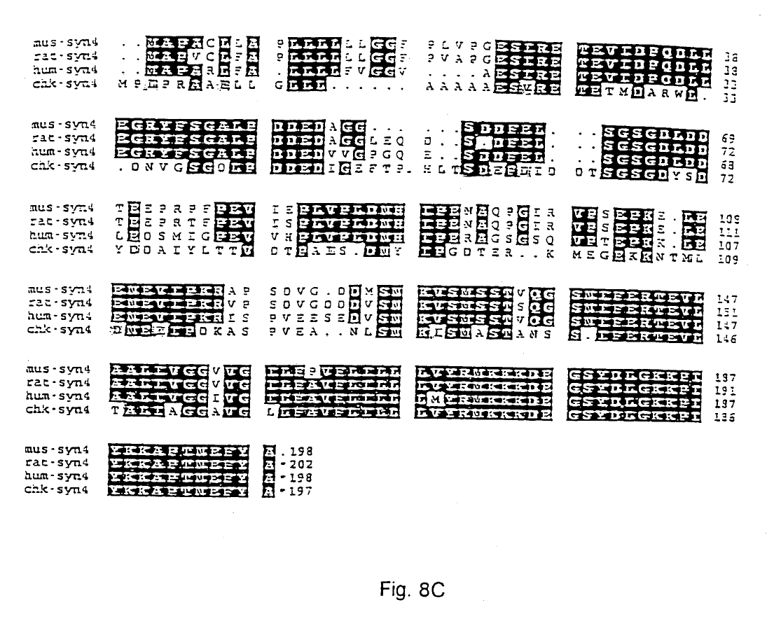

Syndecan-1, -2, -3 and -4 mediate selective binding of FGFs to FGF receptors—The ability of several FGFs to interact with FGF receptors when immobilized on Syn-1, -2, -3 and -4-Fc was compared to their capacity to form specific FGF/FGFR complexes on heparin sepharose. Syn4-Fc preferentially promotes the interaction of bFGF with FGFR1, and with about 2 fold less to FGR2, as measured by alkaline phosphatase activity and cross-linking of the receptors to radio-labeled bFGF. A similar activity was found for aFGF. Most interestingly, high affinity binding of FGF4, FGF7 or FGF9, to their related FGFRs is not enhanced by Syn4-Fc. In contrast, all FGFs tested, demonstrated a high affinity receptor binding when immobilized on heparin sepharose. These results (summarized in Table 2 for Syn4-Fc and in FIG. 6 for Syn1-Fc, Syn2-Fc and Syn4-Fc) directly demonstrate that specific modulators of FGFs.

TABLE 2
Specificity of Syn4-Fc as a modulator of FGFs interactions. Heparin
sepharose or conditioned medium (100 μl) from 293 Syn4-Fc clone #1
immobilized on protein-A sepharose were incubated with 75 ng of the
indicated factor. After washes with HNTG the beads were further
incubated for 2 hours with the indicated FGFR-AP fusion protein and
the bound receptors were determined after extensive washes according
to the AP enzymatic activity.

| FGF | FGFR | Heparin | Syn 4-Fc |
|---|---|---|---|
| 1 | 1 | +++ | +++ |
| 1 | 2 | +++ | ++ |
| 1 | 2IIIb | +++ | + |
| 1 | 3 | ++ | + |
| 2 | 1 | +++ | +++ |
| 2 | 2 | +++ | ++ |
| 4 | 1 | +++ | − |
| 4 | 2 | +++ | − |
| 4 | 2IIIb | +++ | − |
| 4 | 3 | +++ | − |
| 7 | 2IIIb | ++ | − |
| 9 | 2 | ++ | − |
| 9 | 3 | +++ | − |

### Example 6

Syn4-Fc promotes FGF1 mediated proliferation of FGFR1 expressing cells—To study the capacity of syndecan-4 to elicit a heparin-dependent biological response to FGF, we made use of the HS deficient pgs-A745-CHO cells, transfected with FGFR1. These cells were previously shown to efficiently bind FGF2 only in the presence of heparin (25). As shown in FIG. 5C, the cells did not respond to FGF1 in the absence of heparin as measured by DNA synthesis. However, upon the addition of either heparin or purified Syn4-Fc, a clear mitogenic response to FGF1 is observed. Incorporation of ³H-thymidine was enhanced at 100 ng/ml of Syn4-Fc similar to the effect of 100 ng/ml heparin (FIG. 5C). Syn4-Fc or heparin alone had no effect. These results clearly indicate that syndecan-4 can substitute for heparin in mediating heparin-mediated dependent cell proliferation.

### Example 7

2-O-sulfated iduronic acid is required for syndecan-4 mediated FGF-receptor binding—It was previously shown that the heparin structure required to promote FGF2-receptor binding consists of highly sulfated oligosaccharides of at least 10 sugar units in length (21, 34, 35). The sulfation at 2-OH of the α-L-iduronic acid is of special importance and is found in heparin and in HS fragments with high affinity for FGF2 and FGF1 (34, 36). To test the role of this specific modification in the activity of the intact proteoglycan, we expressed Syn4-Fc in Pgs-F17 cells, a mutant CHO cell line deficient of 2-O-sulfotransferase (37). Syn4-Fc produced by these cells had a lower molecular weight (FIG. 6A) and a dramatically reduced affinity towards FGF2. FGF2 pre-bound to heparin, Syn4-Fc or Pgs-F17 made Syn4-Fc, eluted at 1.5, 0.9 and 0.5 M NaCl, respectively (not shown). No binding of FR1-AP to either FGF2 or FGF1 was observed when tested in the presence of the 2-O-sulfate deficient syndecan-4 compared to 293T derived syndecan-4-proteoglycan (FIG. 6B), despite comparable levels of Syn4-Fc core protein. Full-length syndecan-4 expressed in Pgs-F17 cells does not support FGF-FGFR binding as well, similar in that respect to syndecan-4 isolated from HS deficient Pgs-A745 cells. Altogether, these results show that 2-O-sulfation of syndecan-4 glycosaminoglycan chains, is crucial for their activity as modulators of FGF-FGFR interactions.

### REFERENCES

1. Basilico, C., and Moscateili, D. (1992) Adv. Cancer Res. 159, 115-165.

2. Yayon, A., Klagsbrun, M., Esko, J. D., Leder, P., and Ornitz, D. M. (1991) Cell 64, 841-848.

3. Rapraeger, A. C., Krufka, A., and Olwin, B. B. (1991) Science 252, 1705-1707.

4. Brickman, Y. G., Ford, M. D., Small, D. H., Bartlett, P. F., and Nurcombe, V. (1995) J Biol Chem 270, 24941-8.

5. Brown, K. J., Hendry, 1. A., and Parish, C. R. (1995) J Cell Biochem 58, 6-14.

6. Mansukhani, A., Dell'Era, P., Moscatelli, D., Kombluth, S., Hanafusa, H., and Basilico, C. (1992) Proc. Natl. Acad. Sci. USA 89, 3305-3309.

7. McKeehan, W. L., and Kan, M. (1994) Mol Reprod Dev 39, 69-81.

8. Ornitz, D. M., Yayon, A., Flangan, J. G., Svahn, C. M., Levi, E., and Leder, P. (1991) Mol. Cell. Biol. 12, 240-247.

9. Savona, C., Chambaz, E. M., and Feige, J. J. (1991) Growth Factors 5, 273-82.

10. Kan, M., Wang, F., Xu, J., Crabb, J. W., Hou, J., and McKeehan, W. L. (1993) Science 259, 1918-21.

11. Gallagher, J. T., Lyon, M., and Steward, W. P. (1986) Biochem. J. 236, 313-325.

12. Noonan, D., Fulle, A., Vallenta, P., Cai, S., Horigen, E., Sasaki, M., Yamada, Y., and Hassel, J. (1991) J. Biol Chem 266, 22939-22947.

13. David, G., Lories, V., Decock, B., Marynen, P., Cassiman, J. -J., and Berghe, H. V. d. (1990) J. Cell Biol. 111, 3165-3167.

14. Marynen, P., Zhang, J., Cassiman, J. -J., Berghé, H. V. d., and David, G. (1989) J. Biol. Chem. 264, 7017-7024.

15. Carey, D. J., Stahl, R. C., Tucker, B., Bendt, K. A., and Cizmeci, S. G. (1994) Exp Cell Res 214, 12-21.

16. Kojima, T., Katsumi, A., Yamazaki, T., Muramatsu, T., Nagasaka, T., Ohsumi, K., and Saito, H. (1996) J Biol Chem 271, 5914-5920.

17. Bernfield, M., Kokenyesi, R., Kato, M., Hinkes, M. T., Spring, J., Gallo, R. L., and Lose, E. J. (1992) Ann. Rev. Cell Biol. 8.

18. Baciu, P. C., and Goetinck, P. F. (1995) Mol Biol Cell 6, 1503-13.

19. Lee, D., Oh, E. S., Woods, A., Couchman, J. R., and Lee, W. (1998) J Biol Chem 273, 13022-9.

20. Oh, E. S., Woods, A., Lim, S. T., Theibert, A. W., and Couchman, J. R. (1998) J Biol Chem 273, 10624-9.

21. Aviezer, D., Levy, E., Safran, M., Svahn, C., Buddecke, E., Schmidt, A., David, G., Vlodavsky, I., and Yayon, A. (1994) J Biol Chem 269, 114-21.

22. Mali, M., Elenius, K., Miettinen, H. M., and Jalkanen, M. (1993) J Biol Chem 268, 24215-22.

23. Aviezer, D., Hecht, D., Safran, M., Eisinger, M., David, G., and Yayon, A. (1994) Cell 79, 1005-1013.

24. Clasper, S., Vekemans, S., Fiore, M., Plebanski, M., Wordsworth, P., David, G., and Jackson, D. G. (1999) J Biol Chem 274, 24113-23.

25. Aviezer, D., and Yayon, A. (1994) Proc Natl Acad Sci USA 91, 12173-7.

26. Castillo, C. J., Colburn, P., and Buonassisi, V. (1987) Biochem. J. 247, 687-693.

27. Nader, H. B., Dietrich, C. P., Buonassisi, V., and Colbrun, P. (1987) Proc. Natl. Acad. Sci. USA 84, 3565-3569.

28. Wuenscher, M. D., Kohler, S., Goebel, W., and Chakraborty, T. (1991) Mol Gen Genet 228, 177-82.

29. Flanagan, J. G., and Leder, P. (1990) Cell 63, 185-194.

30. Hecht, D., Gray, T. E., Avivi, A., Hasharoni, A., Zimmerman, N., Ma, Y. -S., Seger, R., Nevo, Z., Givol, D., and Yayon, A. (1995).

31. Yayon, A., Zimmer, Y., Guo-Hong, S., Avivi, A., Yarden, Y., and Givol, D. (1992) EMBO J. 11, 1885-1890.

32. Farndale, R. W., Buttle, D. J., and Barrett, A. J. (1986) Biochim Biophys Acta 883, 173-7.

33. Asundi, V. K., and Carey, D. J. (1995) J Biol Chem 270, 26404-26410.

34. Sudhalter, J., Folkman, J., Svahn, C. M., Bergendal, K., and D'Amore, P. A. (1989) J Biol Chem 264, 6892-7.

35. Walker, A., Turnbull, J. E., and Gallagher, J. T. (1994) J Biol Chem 269, 931-5.

36. Guimond, S., Maccarana, M., Olwin, B. B., Lindahl, U., and Rapraeger, A. C. (1993) J Biol Chem 268, 23906-14.

37. Bame, K. J., Zhang, L., David, G., and Esko, J. D. (1994) Biochem J.

38. Spivak, K. T., Lemmon, M. A., Dikio, I., Ladbury, J. E., Pinchasi, D., Huang, J., Jaye, M., Crumley, G., Schlessinger, J., and Lax, I. (1994) Cell 79, 1015-1024.

39. Bonneh-Barkay, D., Shlissel, M., Berman, B., Shaoul, E., Admon, A., Vlodavsky, I., Carey, D. J., Asundi, V. K., Reich-Slotky, R., and Ron, D. (1997) J Biol Chem 272, 12415-21.

40. Filla, M. S., Dam, P., and Rapraeger, A. C. (1998) J Cell Physiol 174, 310-21.

41. Guillonneau, X., Tassin, J., Berrou, E., Bryckaert, M., courtois, Y., and Mascarelli, F. (1996) J Cell Physiol 166, 170-187.

42. Halaban, R., Kwon, B. S., Ghosh, S., Delli, B. P., and Baird, A. (1988) Oncogene Res3, 177-86.

43. Rogelj, S., Weinberg, R. A., Fanning, P., and Klagsbrun, M. (1988) Nature 331, 173-5.

44. Yayon, A., and Kiagsbrun, M. (1990) Proc. Natl. Acad. Sci. USA 87, 5346-5350.

45. Kim, C. W., Goldberger, O. A., Gallo, R. L., and Bernfield, M. (1994) Mol Biol Cell 5, 797-805.

46. Maccarana, M., Casu, B., and Lindahl, U. (1993) J Biol Chem 268, 23898-23905.

47. Turnbull, J. E., Fernig, D. G., Ke, Y., Wilkinson, M. C., and Gallagher, J. T. (1992) J. Biol Chem. 267, 10337-10341.

48. Woods, A., and Couchman, J. R. (1994) Mol Biol Cell 5, 183-92.

49. Couchman, J. R., Austria, R., Woods, A., and Hughes, R. C. (1988) J Cell Physiol 136, 226-36.

50. Habuchi, H., Suzuki, S., Saito, T., Tamura, T., Harada, T., Yoshida, K., and Kimata, K. (1992) Biochem J.

51. Plopper, G. E., McNamee, H. P., Dike, L. E., Bojanowski, K., and Ingber, D. E. (1995) Mol Biol Cell 6, 1349-65.

52. Cizmeci-Smith, G., Langan, E., Youkey, J., Showalter, L. J., and Carey, D. J. (1997) Arterioscier Thromb Vasc Biol 17, 172-80.

1. A molecule capable of promoting high affinity binding of a fibroblast growth factor (FGF) to a FGF receptor (FGFR), said molecule being selected from:
(i) a recombinant chimeric fusion molecule comprising the extracellular domain of a syndecan or a fragment thereof fused to a tag suitable for proteoglycan purification, said fusion molecule being post-translationally glycosylated to carry at least one chain of a heparan sulfate having at least one highly sulfated domain;
(ii) a DNA sequence encoding a chimeric fusion molecule comprising the extracellular domain of a syndecan or a fragment thereof fused to a tag suitable for proteoglycan purification; and
(iii) a sugar molecule from a syndecan carrying at least one chain of a heparan sulfate having at least one highly sulfated domain.

2. A molecule according to claim 1, wherein said molecule promotes high affinity binding of FGF1 and FGF2 to FGFR1.

3. A molecule according to claim 1, wherein said molecule promotes high affinity binding of FGF9 to FGFR2 and to FGFR3.

4. A molecule according to claims 1-3, wherein said extracellular domain is an extracellular domain of syndecan-1, -2, -3 or -4.

5. A molecule according to claim 4, wherein said extracellular domain comprises the glycosylation sites of the syndecan molecule.

6. A molecule according to claim 5, wherein said extracellular domain comprises the amino acids 1-145 of syndecan-4.

7. A molecule according to claim 1 wherein said fragment comprises at least 75 amino acids of the extracellular domain of syndecan-4.

8. A molecule according to claims 1-7, wherein the syndecan extracellular domain is fused to a tag selected from the Fc region of the gamma globulin heavy chain, glutathione S-transferase (GST) or polyHis.

9. A molecule according to claims 1-8, wherein said post-translational glycosylation is carried out in mammalian cells.

10. A molecule according to claim 9, wherein said mammalian cells are selected from endothelial, fibroblast, epithelial cells.

11. A molecule according to claim 10, wherein said mammalian cell is an embryonic kidney cell, an ovary cell or an aortic endothelial cell.

12. A molecule according to claim 11, wherein said mammalian ovarian cells are CHO cells.

13. A molecule according to any one of claims 1-12, wherein said heparan sulfate has at least one highly O-sulfated domain of at least 10 sugar units.

14. A recombinant chimeric fusion molecule comprising the extracellular domain of syndecan-1, -2, -3 or -4 fused to the recombinant Fc region of the gamma globulin heavy chain, carrying at least one chain of a heparan sulfate having at least one highly sulfated domain, herein designated Syn1-Fc, Syn2-Fc, Syn3-Fc and Syn-4Fc, respectively.

15. A pharmaceutical composition comprising a pharmaceutically acceptable carrier and a molecule according to any one of claims 1-14.

16. A pharmaceutical composition according to claim 15, for modulating heparin-dependent growth factor activity relevant for promoting tissue-specific cell proliferation, migration and differentiation.

17. A pharmaceutical composition according to claim 16, wherein said growth factor which activity is modulated is selected from a FGF, a vascular endothelial growth factor (VEGF), hepatocyte growth factor (HGF), an epidermal growth factor (EGF) and keratinocyte growth factor (KGF).

18. A pharmaceutical composition according to claims 15-17, for induction of angiogenesis, bone fracture healing, enhancement of wound healing, promotion of tissue regeneration and treatment of ischemic heart diseases and peripheral vascular diseases.

19. A pharmaceutical composition according to claim 18, for promoting liver regeneration, or for promoting tissue regeneration after transplantation of myocytes into heart tissues, or after transplantation of cells into brain tissue.

20. A pharmaceutical composition according to claims 15-19, wherein said molecule is administered together with a compound selected from a FGF such as FGF2, a VEGF, an EGF, HGF and/or KGF.

21. A pharmaceutical composition according to claim 20, wherein said molecule is administered together with FGF2 for treatment of heart failure by transplantation of myocytes or for promotion of tissue regeneration after transplantation of dopaminergic/neuronal cells.

22. A pharmaceutical composition according to claim 20, wherein said molecule is administered together with FGF2 and/or VEGF for induction of angiogenesis or for treatment of ischemic heart disease or peripheral vascular disease.

23. A pharmaceutical composition according to claim 20, wherein said molecule is administered together with hepatocyte growth factor for promoting liver regeneration.

24. A pharmaceutical composition according to claim 20, wherein said molecule is administered together with keratinocyte growth factor for enhancement of wound healing.

25. Use of a molecule according to any one of claims 1-14, for modulating heparin-dependent growth factor activity relevant for promoting tissue-specific cell proliferation, migration and differentiation.

26. Use according to claim 25, wherein the growth factor is a FGF, a VEGF, an EGF, HGF or KGF.

27. Use according to claims 25-26 for induction of angiogenesis, bone fracture healing, enhancement of wound healing, promotion of tissue regeneration and treatment of ischemic heart diseases and peripheral vascular diseases.

28. Use according to claims 25-27, for promoting liver regeneration, or for promoting tissue regeneration after transplantation of myocytes into heart tissues, or after transplantation of dopaminergic cells into brain tissue.

29. Use according to any one of claims 25-28, together with a compound selected from a FGF such as FGF2, a VEGF, an EGF, HGF and/or KGF.

30. Use according to claim 29, together with FGF2 for treatment of heart failure by transplantation of myocytes or for promotion of tissue regeneration after transplantation of dopaminergic cells.

31. Use according to claim 29, together with FGF2 and/or VEGF for induction of angiogenesis or for treatment of ischemic heart disease or peripheral vascular disease.

32. Use according to claim 29, together with HGF for promoting liver regeneration.

33. Use according to claim 29, together with KGF for enhancement of wound healing.

34. All products, processes and compositions of the invention, as described.


SEQUENCE LISTING (12 sequences)

SEQ ID NO: 1 - DNA, 1327 bp, Mus musculus
aggcgcttgatatcgaattccggaattccggaattccggaattccggaattccgctgttgaagccatggcgcctgcctgcctgcttgcgccgctgctgctgctgctcctcggaggcttccccttggtccccggagagtcgattcgagagacagaggtcatcgacccccaggacctcctggaaggcagatacttctctggagccctccccgacgacgaagatgctggcggctcggatgactttgagctctcgggttctggagatctggatgacacggaggagcccaggcccttccctgaagtgattgagcccttggtgccactggataaccacatccctgagaatgcacagcctggcatccgtgtcccctcagagcccaaggaactggaagagaatgaggtcattcctaaaagggccccctccgacgtgggggatgacatgtccaacaaagtatccatgtccagcactgtccagggcagcaacatctttgagagaactgaggtcttggcagctctgatcgtgggcggcgtggtaggcatcctctttcccgttttcctgatcctgctgctggtgtaccgcatgaagaagaaggacgaaggcagttacgacttgggcaagaaacccatctacaaaaaagcccccaccaaggagttctacgcatgaagcttcctcccgcgagcgctgcttggacttattggggagaggagtggagggttgtgggtggcgggcgttggcagagagcagcaggcaccttaatgctgacttgtcagtatctccatctctagtcacctttctggtgtcagaagagatgtgatcttctactgtgctgcctgagagagagagagagagagagagagagagagagagaggggctgtgtctgtgtgtctgtgtctcagttgctctggcagaaaaatggggttaaacttgccctttctgaaggcaagcctacaattgggtcttttgttgtcattgttccaaatttccagaaatagaatataggaccagtttagatcctgtagtaaacatgtcccatctatgactgccttgattatagaggcaaggggttactgtgtgaatcccggctcccttccacatgctgtacaccctatccatctgtcaggagctggggcaaggagccaaaccctctgcaccttgagatgagtctgccatgagaacttgctcacgctgcagagtctctgtggcgtacctgggggcattctaagtccagtgacttttgaaattcaacctttaaaaaaaaaaaaatctagggagggcggggtggagtgctgaaagctcacactgaagtgtgtttggatgctctgaacta

SEQ ID NO: 2 - protein, 198 aa, Mus musculus
MAPACLLAPLLLLLLGGFPLVPGESIRETEVIDPQDLLEGRYFSGALPDDEDAGGSDDFELSGSGDLDDTEEPRPFPEVIEPLVPLDNHIPENAQPGIRVPSEPKELEENEVIPKRAPSDVGDDMSNKVSMSSTVQGSNIFERTEVLAALIVGGVVGILFPVFLILLLVYRMKKKDEGSYDLGKKPIYKKAPTNEFYA

SEQ ID NO: 3 - protein, 311 aa, Mus musculus
MRRAALWLWLCALALRLQPALPQIVAVNVPPEDQDGSGDDSDNFSGSGTGALPDTLSRQTPSTWKDVWLLTATPTAPEPTSSNTETAFTSVLPAGEKPEEGEPVLHVEAEPGFTARDKEKEVTTRPRETVQLPITQRASTVRVTTAQAAVTSHPHGGMQPGLHETSAPTAPGQPDHQPPRVEGGGTSVIKEVVEDGTANQLPAGEGSGEQDFTFETSGENTAVAAVEPGLRNQPPVDEGATGASQSLLDRKEVLGGVIAGGLVGLIFAVCLVAFMLYRMKKKDEGSYSLEEPKQANGGAYQKPTKQEEFYA

SEQ ID NO: 4 - protein, 211 aa, Rattus norvegicus
MRVRATSPGNMQRAWILLTLGLMACVSAETRAELTSDKDMYLDSSSIEEASGLYPIDDDDYSSASGSGAYEDKGSPDLTTSQLIPRISLTSAAPEVETMTLKTQSITPTQTESPEETDKKEFEISEAEEKQDPAVKSTDVYTEKHSDNLFKRTEVLAAVIAGGVLGFLFAIFLILLLVYRMRKKDEGSYDLGERKPSSAAYQKAPTKEFYA

SEQ ID NO: 5 - protein, 353 aa, Mus musculus
LRETAMRFIPDIALAAPTAPAMLPTTVIQPVDTPFEELLSEHPGPEPVTSPPLVTEVTEVVEEPSQRATTISTTTSTTAATTTGAPTMATAPATAATTAPSTPAAPPATATTADIRTTGIQGLLPLPLTTAATAKATTPAVPSPPTTVTTLDTEAPTPRLVNTATSRPRALPRPVTTQEPEVAERSTLPLGTTAPGPTEVAQTPTPESLLTTTQDEPEVPVSGGPSGDFELQEETTQPDTANEVVAVEGAAAKPSPPLGTLPKGARPGLGLHDNAIDSGSSAAQLPQKSILERKEVLVAVIVGGVVGALFAAFLVTLLIYRMKKKDEGSYTLEEPKQASVTYQKPDKQEEFYA

SEQ ID NO: 6 - protein, 202 aa, Rattus norvegicus
MAPVCLFAPLLLLLLGGFPVAPGESIRETEVIDPQDLLEGRYFSGALPDDEDAGGLEQDSDFELSGSGDLDDTEEPRTFPEVISPLVPLDNHIPENAQPGIRVPSEPKELEENEVIPKRVPSDVGDDDVSNKVSMSSTSQGSNIFERTEVLAALIVGGVVGILFAVFLILLLVYRMKKKDEGSYDLGKKPIYKKAPTNEFYA

SEQ ID NO: 7 - protein, 198 aa, Homo sapiens
MAPARLFALLLLFVGGVAESIRETEVIDPQDLLEGRYFSGALPDDEDVVGPGQESDDFELSGSGDLDDLEDSMIGPEVVHPLVPLDNHIPERAGSGSQVPTEPKKLEENEVIPKRISPVEESEDVSNKVSMSSTVQGSNIFERTEVLAALIVGGIVGILFAVFLILLLMYRMKKKDEGSYDLGKKPIYKKAPTNEFYA

SEQ ID NO: 8 - protein, 197 aa, Gallus gallus
MPLPRAAFLLGLLLAAAAAESVRETETMDARWLDNVGSGDLPDDEDIGEFTPHLTSDEFDIDDTSGSGDYSDYDDAIYLTTVDTPAISDNYIPGDTERKMEGEKKNTMLDNEIIPDKASPVEANLSNKISMASTANSSIFERTEVLTALIAGGAVGLLFAVFLILLLVYRMKKKDEGSYDLGKKPIYKKAPTNEFYA

SEQ ID NO: 9 - DNA, 28 bp, Artificial sequence
  synthetic oligonucleotide
cccaagctttgtgctgttggaaccatgg

SEQ ID NO: 10 - DNA, 27 bp, Artificial sequence
  synthetic oligonucleotide
gcggatccgcctcatgcgtagaactcg

SEQ ID NO: 11 - DNA, 26 bp, Artificial sequence
  synthetic oligonucleotide
cgggatcctcagttctctcaaagatg

SEQ ID NO: 12 - protein, 11 aa, Artificial sequence
  synthetic peptide
SYDLGKKPIYK